(19) United States
(12) Patent Application Publication
(10) Pub. No.: US 2024/0398365 A1
(43) Pub. Date: Dec. 5, 2024

(54) MOBILE RADIOGRAPHIC IMAGING APPARATUS, DISPLAY METHOD, AND RECORDING MEDIUM

(71) Applicant: Konica Minolta, Inc., Tokyo (JP)

(72) Inventor: Takuya YAMAMURA, Tokyo (JP)

(21) Appl. No.: 18/675,960

(22) Filed: May 28, 2024

(30) Foreign Application Priority Data
May 30, 2023 (JP) 2023-088486

(51) Int. Cl. A61B 6/46; A61B 6/00

(52) U.S. Cl. CPC A61B 6/463; A61B 6/4405

(57) ABSTRACT

Disclosed is a mobile radiographic imaging apparatus, including: a hardware processor that acquires a moving image composed of a series of frames from an imaging pert that captures the moving image by irradiating a subject with radiation; and a display part that displays a display image based on the moving image. The display port displays a first specific frame in a former portion of the series of frames and a second specific frame in a latter portion of the series of frames as still images.

### CROSS-REFERENCE TO RELATED APPLICATIONS

The entire disclosure of Japanese Patent Application No. 2023-088486, filed on May 30, 2023, including description, claims, drawings and abstract is incorporated herein by reference.

### BACKGROUND OF THE INVENTION

### Technical Field

The present invention relates to a mobile radiographic imaging apparatus, a display method, and a recording medium.

### Description of Related Art

Mobile radiographic imaging apparatus have been known to take radiographs in locations other than radiography rooms in hospitals (e.g., hospital rooms, operating rooms, and the like). In recent years, mobile radiographic imaging apparatus capable of taking moving images have also been put to practical use.

The present inventors have found that the preview image displayed on a mobile radiographic imaging apparatus capable of taking moving images may provide new value by using the preview image for other purposes than simply confirming whether or not a retake is necessary. For example, instead of fluoroscopic imaging, which requires a large amount of radiation exposure and a trip to the imaging room, a mobile radiographic imaging apparatus, which has a small amount of radiation exposure and can record images in the patient's room, can be used to record moving images, and procedures can be performed while viewing and checking the preview images obtained. Confirmation of the procedure includes, for example, checking whether the catheter/feed tube has been inserted correctly and whether there is any residual material (e.g., gauze) after the procedure.

However, unlike ordinary fluoroscopy systems, a flat panel detector (FPD) used in mobile radiographic imaging apparatus has a large angle of view and generates a huge amount of image data. Taking into account infection prevention and ease of use, image transfer from the FPD needs to be wireless, and the image transfer takes time. This makes it difficult to display images in real time even when communication conditions are good.

For example, JP 2018-157869A describes a radiographic image display apparatus that generates and displays a moving image for preview when image data for each frame of the moving image is transferred. The moving image for preview is an image with a frame rate lower than the frame rate of the actual moving image capture, which has undergone simpler image processing than the moving image to be displayed as the main image.

### SUMMARY OF THE INVENTION

However, in JP 2018-157869A, the generated moving image for preview is displayed according to the order in which they were taken. Therefore, even if the moving image capture is completed, the latter portion of frames cannot be displayed immediately. In particular, if the transfer time from the FPD is long, it takes a considerable amount of time before the latter portion of the frames is displayed. Therefore, even after the moving image capture is completed, a user cannot early check how a subject has changed during the image capture. For example, when a catheter insertion procedure is captured as a moving image, it is not possible for the user to confirm, in an early stage after completion of the capture, whether the catheter has been inserted in the correct position.

Objects of the present invention include allowing a user to promptly check, after completion of moving image capture, how a subject has changed during the moving image capture in a mobile radiographic imaging apparatus.

To achieve at least one of the abovementioned objects, according to an aspect of the present invention, a mobile radiographic imaging apparatus reflecting one aspect of the present invention comprises: a hardware processor that acquires a moving image composed of a series of frames from an imaging part that captures the moving image by irradiating a subject with radiation; and a display part that displays a display image based on the moving image, wherein the display part displays a first specific frame in a former portion of the series of frames and a second specific frame in a latter portion of the series of frames as still images.

To achieve at least one of the abovementioned objects, according to another aspect of the present invention, a display method performed by a mobile radiographic imaging apparatus comprises; acquiring a moving image composed of a series of frames from an imaging part that captures the moving image by irradiating a subject with radiation; and displaying a display image based on the moving image by a display part, wherein, in the displaying, a first specific frame in a former portion of the series of frames and a second specific frame in a latter portion of the series of frames are displayed as still images by the display part.

To achieve at least one of the abovementioned objects, according to yet another aspect of the present invention, a non-transitory computer-readable recording medium stores a program, wherein the program causes a computer of a mobile radiographic imaging apparatus to function as: an image acquirer that acquires a moving image composed of a series of frames from an imaging part that captures the moving image by irradiating a subject with radiation; and a display part that displays a display image based on the moving image, wherein the display part displays a first specific frame in a former portion of the series of frames and a second specific frame in a latter portion of the series of frames as still images.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a diagram illustrating an example of the entire structure of a mobile radiographic imaging apparatus;

FIG. 2 is a block diagram illustrating a functional configuration of the mobile radiographic imaging apparatus;

FIG. 3 is a block diagram illustrating a functional configuration of an FPD;

FIG. 4 illustrates a display example of an imaging screen;

FIG. 5 is a flowchart illustrating moving image transfer processing executed by a controller of an FPD;

FIG. 6 is a flowchart illustrating moving image display processing executed by a controller of a main body;

FIG. 7 illustrates a display example of an imaging screen in step S22 of FIG. 6;

FIG. 8 illustrates a display example of an imaging screen in step S25 of FIG. 6;

FIG. 9 illustrates a display example of an imaging screen in step S27 of FIG. 6;

FIG. 10 illustrates a display example of an imaging screen on which the first frame, a key frame, and the final frame are displayed side by side;

FIG. 11 illustrates a display example of an imaging screen in Modification Example 2; and

FIG. 12 illustrates a display example of an imaging screen in Modification Example 6.

### DETAILED DESCRIPTION

Hereinafter, one or more embodiments of the present invention will be described with reference to the drawings. However, the embodiments described below are subject to various technically preferred limitations for implementing the present invention. Therefore, the scope of the invention is not limited to the disclosed embodiments.

### Configuration of Mobile Radiographic Imaging Apparatus

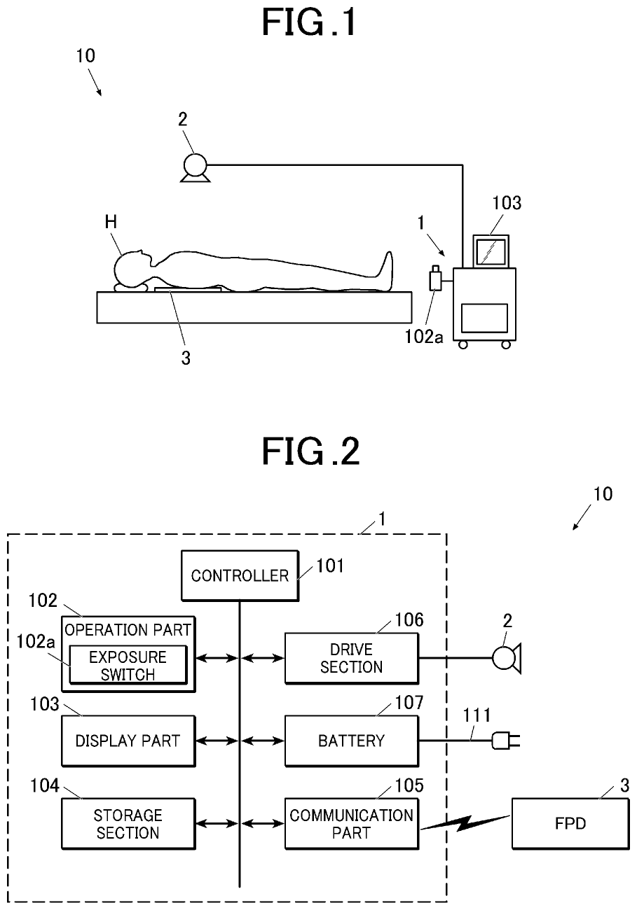

FIG. 1 is a diagram illustrating an example of the entire structure of a mobile radiographic imaging apparatus 10 in the present embodiment. The mobile radiographic imaging apparatus 10 is, for example, an apparatus for performing radiography on rounds for patients who are difficult to move. The mobile radiographic imaging apparatus 10 includes a main body 1, a radiation source 2, and an FPD 3. The mobile radiographic imaging apparatus 10 includes wheels in the main body 1 and is configured as a movable medical vehicle. The mobile radiographic imaging apparatus 10 may be a portable apparatus that does not have wheels.

The mobile radiographic imaging apparatus 10 captures a still image or a moving image of a subject H by irradiating the subject with radiation from the radiation source 2 with the FPD 3 positioned opposite the radiation source 2 across the subject H. In the present embodiment, the still image capture refers to acquisition of a single image of a subject in response to a single capture operation (pressing the exposure switch 102a). The moving image capture refers to acquisition of a plurality of images of a subject by irradiating the subject with pulsed irradiation of X-rays or other radiation in response to a single capture operation. The pulsed irradiation refers to repeated irradiation of radiation in the form of pulses at predetermined time intervals. Alternatively, the moving image capture refers to acquisition of a plurality of images of a subject by continuously irradiating the subject with radiation. The continuous irradiation refers to continuous, uninterrupted irradiation of radiation at a low dose rate. A series of images obtained by the moving image capture is referred to as a moving image (video). Each of the plurality of images that constitute a moving image is referred to as a frame. The moving image capture includes dynamic image capture that acquires a dynamic image consisting of a plurality of frames indicating a dynamic state of a subject.

FIG. 2 is a block diagram illustrating a functional configuration of the mobile radiographic imaging apparatus 10.

The main body 1 of the mobile radiographic imaging apparatus 10 has a function as a console (imaging controller).

As shown in FIG. 2, the main body 1 includes a controller (hardware processor) 101, an operation part 102, a display part 103, a storage section 104, a communication part 105, a drive section 106, a battery 107, and the like. Each unit of the main body 1 is connected by a bus.

The controller 101 includes a central processing unit (CPU), a random access memory (RAM), and the like. The CPU of the controller 101 reads a system program and various processing programs stored in the storage section 104, develops the programs in the RAM, and executes various processes in accordance with the developed programs. The controller 101 functions as a hardware processor, an image processor, and a notifier of the present invention by executing moving image display processing described below.

The operation part 102 includes a touch screen or the like in which transparent electrodes are arranged in a lattice shape so as to cover the surface of the display part 103. The touch screen detects the position pressed by a finger, a touch pen, or the like, and inputs the position information as operation information to the controller 101. The operation part 102 includes an exposure switch 102a. The exposure switch 102a is used by the user to start irradiation.

The display part 103 includes a monitor such as a liquid crystal display (LCD) or a cathode ray tube (CRT). The display part 103 displays screens in accordance with an instruction of a display signal input from the controller 101.

The storage section 104 includes a nonvolatile semiconductor memory, a hard disk, or the like. The storage section 104 stores various programs executed by the controller 101, parameters required to execute processing by the programs, and data such as processing results.

The storage section 104 also stores examination order information. The examination order information includes patient information and examination information. The patient information includes the patient ID, name, sex, age, hospital room (ward), and the like of the patient to be examined. The examination information includes the examination ID, examination date, and an imaging order for each image capture performed at the examination. The imaging order includes an imaging part, an imaging direction, the classification of still image capture or moving image capture, and the like.

Furthermore, the storage section 104 is provided with a temporary storage area for temporarily storing images transferred from the FPD 3 and images that have been processed.

The communication part 105 transmits and receives data to and from the FPD 3 or an external device. In the present embodiment, the communication part 105 includes an antenna, and transmits and receives data to and from the FPD 3 or the like by wireless communication via an access point (not illustrated). The communication part 105 may be configured to transmit and receive data by wired communication. The communication part 105 functions as an image acquirer.

The drive section 106 is a circuit that drives the tube of the radiation source 2. The drive section 106 and the radiation source 2 are connected to each other via a cable.

The battery 107 supplies electric power to each part of the main body 1 and the radiation source 2. The battery 107 can be charged externally via an AC cable 111.

The radiation source 2 is driven by the drive section 106 to irradiate the subject H with radiation (X-rays).

The FPD 3 is an imaging apparatus (imaging part) that supports still and moving image capture. As shown in FIG. 1, the FPD 3 is positioned opposite the radiation source 2 with the subject H interposed therebetween. The FPD 3 generates electric charges in accordance with radiation transmitted through the subject H from the radiation source 2. The generated electric charges are read out as image data, and thereby a radiation image is captured.

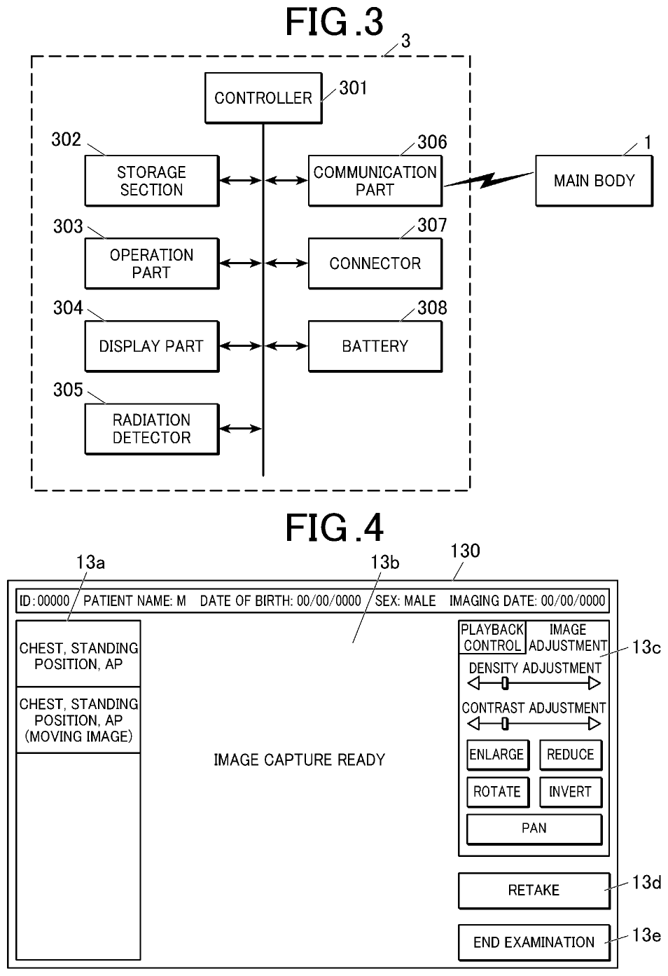

FIG. 3 is a block diagram illustrating a functional configuration of the FPD 3. As illustrated in FIG. 3, the FPD 3 includes a controller 301, a storage section 302, an operation part 303, a display part 304, a radiation detector 305, a communication part 306, a connector 307, a battery 308, and the like. Each unit of the FPD 3 is connected by a bus.

The controller 301 includes a CPU, a RAM, and the like. The controller 301 reads out various programs stored in the storage section 302, develops the programs in the RAM, and centrally controls each unit of the FPD 3 according to the developed programs.

The storage section 302 includes a nonvolatile semiconductor memory or the like. The storage section 302 stores various programs executed by the controller 301, parameters required to execute processing by the programs, and data such as processing results.

The storage section 302 temporarily stores image data (frames) awaiting transfer to the main body 1.

The operation part 303 includes various operation switches such as a power switch. The operation part 303 outputs operation signals to the controller 301 according to user operations on various switches.

The display part 304 is provided, for example, on the side of the housing of the FPD 3, and displays various information under control from the controller 301.

The radiation detector 305 includes, for example, radiation detection elements arranged in a two-dimensional array on a glass substrate. Each radiation detection element is composed of a semiconductor image sensor such as a photodiode. The radiation detection element detects radiation emitted from the radiation source 2 and transmitted through at least the subject H in accordance with its intensity, converts the detected radiation into an electric signal, and accumulates the electric signal. For example, each radiation detection element is connected to a switching unit such as a thin film transistor (TFT). The switching unit acquires image data by controlling accumulation and readout of electric signals in the radiation detection element. The radiation detector 305 can be either an indirect conversion type in which radiation is converted into electric signals by photoelectric conversion elements via a scintillator, or a direct conversion type in which radiation is directly converted into electric signals.

The communication part 306 includes an antenna and transmits and receives data to and from the main body 1 by wireless communication.

The connector 307 is a connection part for connecting to external devices directly or via a cable.

The battery 308 accumulates (charges) electric power supplied from an external device, such as a cradle or an imaging table, connected thereto via the connector 307, and supplies the electric power to each unit of the FPD 3.

### Operation of Mobile Radiographic Imaging Apparatus

The operation of the mobile radiographic imaging apparatus 10 is described next.

To start radiation image capture, the user, the person who performs the radiation image capture, operates the operation part 102 of the main body 1 to display an examination list screen (not illustrated) and selects an examination. The controller 101 displays the imaging screen 130 corresponding to the selected examination by the display part 103.

FIG. 4 is a diagram illustrating an example of the imaging screen 130. As illustrated in FIG. 4, the imaging screen 130 includes an imaging order list 13a, an image display field 13b, an image adjustment field 13c, a retake button 13d, an end examination button 13e, and the like.

The imaging order list 13a is a list of imaging orders included in the examination order information of the selected examination. The imaging order includes an imaging part, an imaging direction, the classification of still image capture or moving image capture, and the like.

The user selects the next imaging order from the imaging order list 13a, and positions the subject H, radiation source 2, and FPD 3.

In the main body 1, when an imaging order is selected by operation of the operation part 102, the controller 101 sets radiation emission conditions to the drive section 106 corresponding to the selected imaging order. The controller 101 also transmits image reading conditions to the FPD 3 corresponding to the selected imaging order by the communication part 105. In a case where the selected imaging order is moving image capture, the radiation emission conditions for the moving image capture are set in the drive section 106 and the image reading conditions for the moving image capture in the FPD 3.

When the image capture preparation is complete, the user presses the exposure switch 102a.

When the first switch of the exposure switch 102a is pressed, the controller 101 of the main body 1 puts the radiation source 2 into standby by the drive section 106. Further, the controller 101 initiates a reset on the FPD 3 via the communication part 105. When the second switch of the exposure switch 102a is pressed, the controller 101 causes the radiation source 2 to start emitting radiation by the drive section 106 in synchronization with the FPD 3 in which the reset is completed, and causes the FPD 3 to start taking a radiation image.

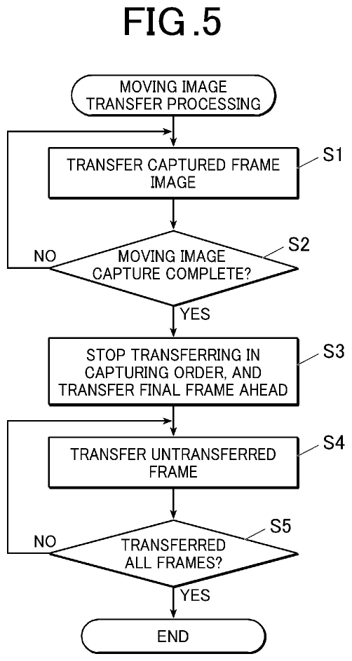

FIG. 5 is a flowchart illustrating the moving image transfer processing executed by the FPD 3 when moving image capture is started. The processing in FIG. 5 is executed by the controller 301 in cooperation with a program stored in the storage section 302.

When the moving image capture is started, the controller 301 sequentially transfers the frames captured by the radiation detector 305 to the main body 1 via the communication part 306 (step S1).

In step S1, the controller 301 sequentially stores the frames captured by the radiation detector 305 in a temporary storage area of the storage section 302 Then, the controller 301 adds a frame number indicating the capturing order to each frame, and transfers the frame to the main body 1 via the communication part 306.

Next, the controller 301 determines whether the moving image capture has been completed (step S2).

Here, the controller 301 determines that the moving image capture is complete when the completion of image capture is notified from the main body 1 or when a predetermined time has elapsed since the start of image capture.

If it is determined that the moving image capture has not been completed (step S2; NO), the processing returns to step S1. The controller 301 transfers the frames captured by the radiation detector 305 to the main body 1 in the capturing order via the communication part 306 until the moving image capture is completed. In the main body 1, the frames transferred from the FPD 3 are sequentially received by the communication part 105 and are displayed on the imaging screen 130 of the display part 103.

If it is determined that the moving image capture is completed (step S2; YES), the controller 301 stops the image transfer in the capturing order, and transfers the final frame captured last to the main body 1 ahead of other untransferred frames via the communication part 306 (step S3).

Here, in a case where frames captured in moving image capture are transferred by wireless communication, the speed of capturing a single new frame may be faster than that of transferring a single frame. In this case, at the time of completion of image capturing, frames captured before the final frame remain in the temporary storage area of the storage section 302 as untransferred frames. In step S3, the controller 301 transmits the final frame to the main body 1 via the communication part 306 ahead of these untransferred frames. When transmitting the final frame to the main body 1, the controller 301 adds information to the final frame indicating that it is the final frame.

Next, the controller 301 resumes the transfer of the untransferred frames stored in the temporary storage area of the storage section 302 via the communication part 306, and sequentially transfers the untransferred frames to the main body 1 (step S4).

When the transfer of all the frames captured by the moving image capture is completed (step S5; YES), the controller 301 ends the moving image transfer processing.

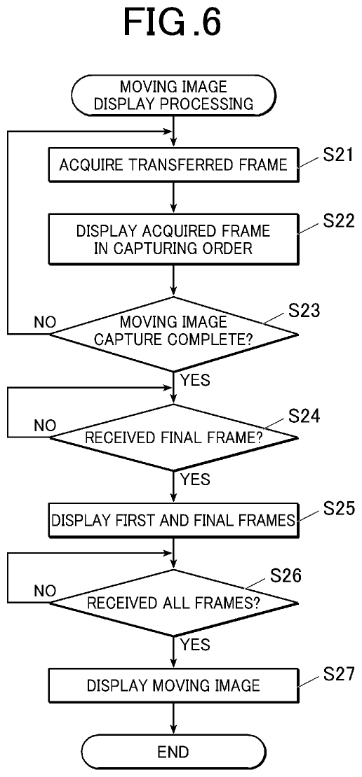

FIG. 6 is a flowchart illustrating the moving image display processing executed by the main body 1 when moving image capture is started. The processing in FIG. 6 is executed by the controller 101 in cooperation with a program stored in the storage section 104.

First, the controller 101 receives (acquires) the frames transferred from the FPD 3 by the communication part 105 and sequentially stores the frames in the temporary storage area of the storage section 104 (step S21).

Next, the controller 101 displays the acquired frames in the capturing order by the display part 103 (step S22).

Specifically, the controller 101 displays the acquired frames in the image display field 13b of the imaging screen 130 in the capturing order, starting with the first frame. At that time, the controller 101 may not display all the frames, but may thin out and display the frames at predetermined frame intervals.

Here, when displaying the acquired frames in the capturing order, the controller 101 performs simpler image processing than that applied to each frame when displaying moving images after the transfer of all the frames is completed. For example, the controller 101 performs offset correction, gain correction, tone correction, and the like among the correction processes performed on each frame displayed as a moving image, and omits defective pixel correction. The controller 101 may also perform the above correction after thinning out the pixels of the acquired frames to increase processing efficiency during image processing.

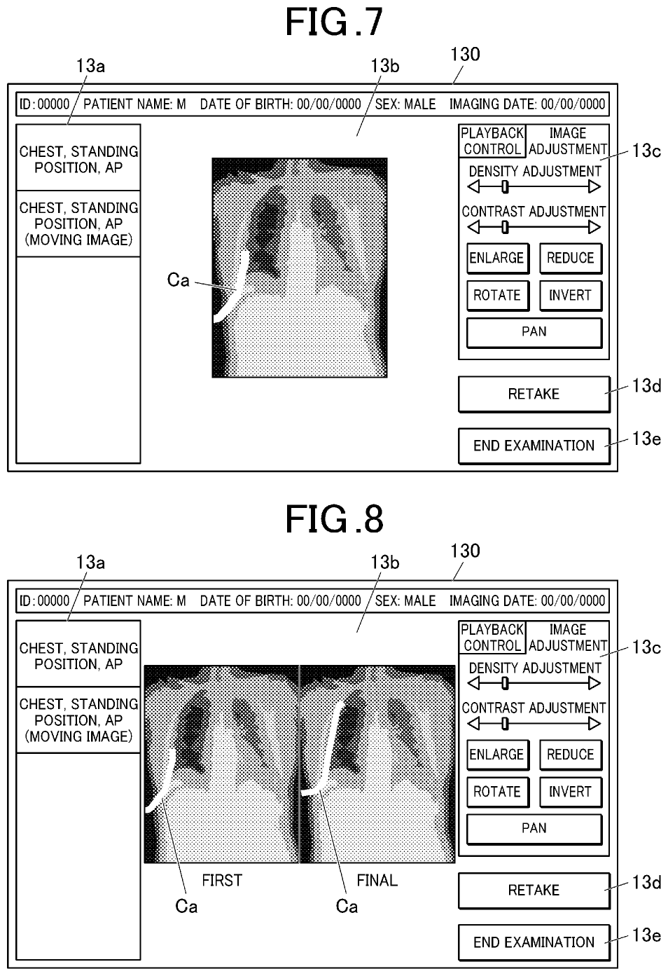

FIG. 7 is a diagram illustrating an image display example of the imaging screen 130 in step S22. In step S22, the frames that have been taken and transferred are sequentially displayed in the image display field 13b. FIG. 7 shows an example of displaying frames obtained by capturing a moving image of the insertion of a catheter Ca.

Next, the controller 101 determines whether the moving image capture has been completed (step 23).

The controller 101 determines that the moving image capture is complete when the exposure switch 102a is released or a predetermined time has elapsed since the start of image capture.

If it is determined that the moving image capture has not been completed (step S23; NO), the processing returns to step S21.

If it is determined that the moving image capture is completed (step S23; YES), the controller 101 waits for the final frame to be received by the communication part 105 (step S24).

When the final frame is received (step S24; YES), the controller 101 displays the first frame and the final frame by the display part 103 (step S25).

Specifically, the controller 101 displays the first frame and the final frame side by side as still images in the image display field 13b of the imaging screen 130. Alternatively, the controller 101 may alternately display the first frame and the final frame as still images in the image display field 13b of the imaging screen 130 at predetermined time intervals or in response to a user operation.

Here, as with the first frame, the controller 101 performs simpler image processing on the final frame than is performed on each frame when displaying a moving image after the transfer of all the frames is complete, and displays the processed images. For example, the controller 101 performs offset correction, gain correction, tone correction, and the like on the final frame, and omits defective pixel correction. The controller 101 may also perform the above correction after thinning out the pixels of the final frame to increase processing efficiency during image processing.

FIG. 8 is a diagram illustrating a display example of the imaging screen 130 in step S25. As shown in FIG. 8, the first frame (First) and the final frame (Final) are displayed side by side in the image display field 13b of the imaging screen 130. In FIG. 8, the first and final frames are shown when the insertion of the catheter Ca in the chest is captured as a moving image.

As described above, when the final frame obtained by the moving image capture is received, the controller 101 displays the first frame and the final frame by the display part 103, as shown in FIG. 8. In this way, the controller 101 displays the final frame without waiting for other frames to be displayed. Therefore, the user can check how the subject H has changed during the moving image capture at an early stage after the completion of the image capture. For example, the screen shown in FIG. 8 allows the user to check at an early stage after the completion of image capture where the catheter Ca was inserted from and whether the catheter Ca has reached the target location. By transferring the final frame prior to the other untransferred frames when the moving image capture is completed on the FPD 3, the first frame and the final frame can be displayed by the display part 103 at an early stage, even if the transfer takes time. Therefore, even if transfer takes time, the user can check how the subject H has changed during the moving image capture at an early stage after the completion of the image capture.

When displaying the first and final frames, the controller 101 may perform image processing on the first and final frames that is different from the image processing applied to each frame when displaying a moving image after all the frames have been transferred. For example, frequency emphasis processing may be performed on the first frame and the final frame to emphasize a predetermined structure. For example, if the moving image is obtained by capturing the insertion of the catheter Ca, frequency emphasis processing may be performed on the first frame and the final frame to emphasize the distal end portion of the catheter Ca. This allows for easy viewing of structures of interest.

Next, the controller 101 determines whether all the frames have been received (step 26). If it is determined that the reception of all the frames has not been completed (step S26; NO), the controller 101 waits for the completion of the reception of all the frames.

If the controller 101 determines that the reception of all the frames is completed (step 26; YES), the controller 130 displays the moving image obtained by the moving image capture by the display part 103 (step S27). Specifically, the controller 101 displays the moving image obtained by the image capture in the image display field 13b of the imaging screen 130. Then, the controller 101 ends the moving image display processing.

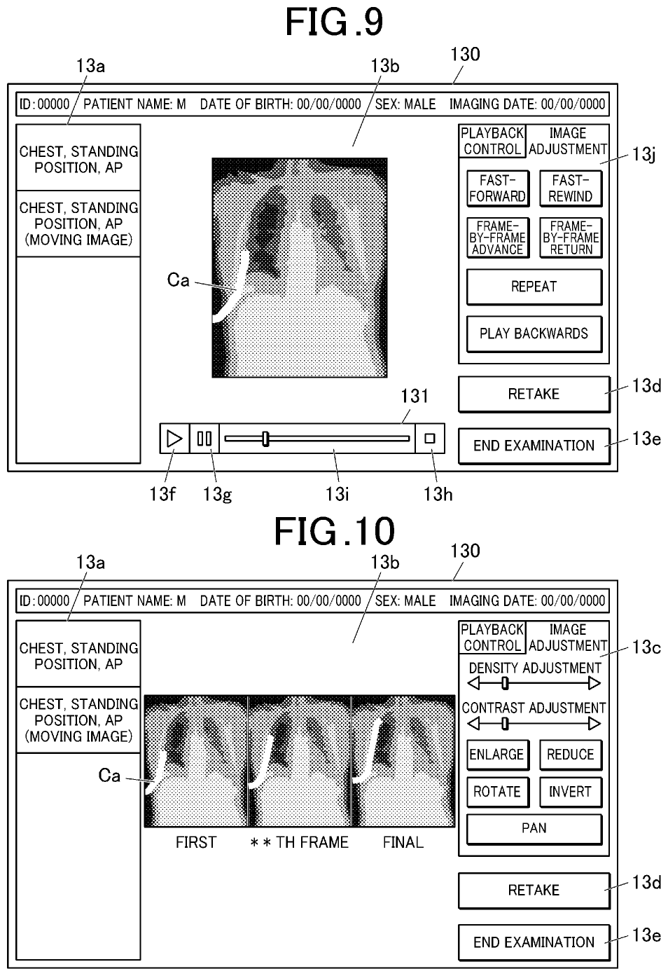

FIG. 9 is a diagram illustrating a display example of the imaging screen 130 in step S27. As shown in FIG. 9, in step S27, a moving image and a playback tool 131 for playing back the moving image are displayed in the image display field 13b of the imaging screen 130. The playback tool 131 has a moving image playback button 13f, a pause button 13g, a stop button 13h, a slide bar 13i, and the like. The image adjustment field 13c is automatically switched to the playback control field 13j for display. The playback control field 13j displays function buttons related to playback that are not included in the playback tool 131, such as a fast-forward button, fast-rewind button, frame-by-frame advance, frame-by-frame return, and repeat playback.

The controller 101 performs image processing for displaying a moving image on each received frame and displays the processed frames in the image display field 13b of the imaging screen 130. Examples of the image processing include offset correction, gain correction, defective pixel correction, tone correction, and frequency emphasis processing. The timing for applying image processing for displaying a moving image to the received frames is not particularly limited.

The user who needs to check the moving image can press the moving image playback button 13f to play and check the moving image. If the user can understand the change in the subject H from the first frame and the final frame displayed in step S25, the user can omit the moving image display and proceed to the next image capture or end the examination.

As described above, when the main body 1 of the mobile radiographic imaging apparatus 10 receives the final frame, the main body 1 displays the first frame and the final frame side by side on the same screen. The main body 1 may also display the first and final frames one at a time by switching between the first and final frames.

Therefore, the user can check how the subject H has changed during the moving image capture at an early stage after the completion of the image capture.

Hereinafter, variations of the above-described embodiment are described.

### Modification Example 1

Although the controller 101 displays the first frame and the final frame side by side or in a switching manner in the above embodiment, a key frame (key image) may also be displayed together. The key frame is a frame that satisfies a predetermined condition of the captured moving image. The condition of the key frame includes, for example, any one of the following:
(1) a frame for which the difference in signal value in a predetermined region from the first frame is equal to or greater than a certain threshold value;
(2) a frame for which the difference in signal value in a predetermined region from an adjacent frame is equal to or greater than a threshold value;
(3) a frame in which a frame-to-frame movement of a predetermined structure is equal to or more than a threshold value;
(4) a frame in which the amount of movement of a predetermined structure relative to the first frame is equal to or greater than a threshold value; or
(5) if the imaging part is the chest, the frame in which the diaphragm is at its lowest (highest) position (maximum expiratory position frame or maximum inspiratory position frame).

The condition of the key frame can be set by a user operation on the operation part 303 or by transmission from the main body 1. The above-described “predetermined area” and “predetermined structure” can also be set by a user operation.

For example, when a frame is generated by the radiation detector 305, the controller 301 determines whether the frame satisfies a predetermined condition of a key frame. If the frame satisfying the key frame condition has not been transferred at the time of completion of image capture, the controller 301 transmits the key frame and the final frame ahead of other untransferred frames. In this step, the controller 301 adds information indicating that it is the key frame to the key frame and transfers the key frame. For (5) above, the controller 301 identifies a frame that satisfies the key frame condition after the completion of image capture. If the frame satisfying the key frame condition has not been transferred at the time of the completion of image capture, the controller 301 transmits the key frame and the final frame ahead of other untransferred frames. In step S25, the controller 101 of the main body 1 displays the first frame, the key frame, and the final frame side by side.

FIG. 10 is a diagram illustrating an example of the imaging screen 130 on which the first frame, the key frame, and the final frame are displayed side by side. As shown in FIG. 10, displaying the first frame, the key frame, and the final frame side by side, allows the user to see not only the first and final frames, but also another frame of interest at an early stage.

The key frame may be identified by the controller 101 of the main body 1 instead of being identified in the FPD 3. For example, under circumstances where a wireless communication environment has been established and the final frame is transferred to the main body 1 almost without delay after completion of image capture, the controller 101 of the main body 1 may identify the key frame. Then, the controller 101 performs image processing on the first frame, the key frame, and the final frame prior to the other frames and displays the processed frames. This allows the user to check a frame of interest at an early stage.

### Modification Example 2

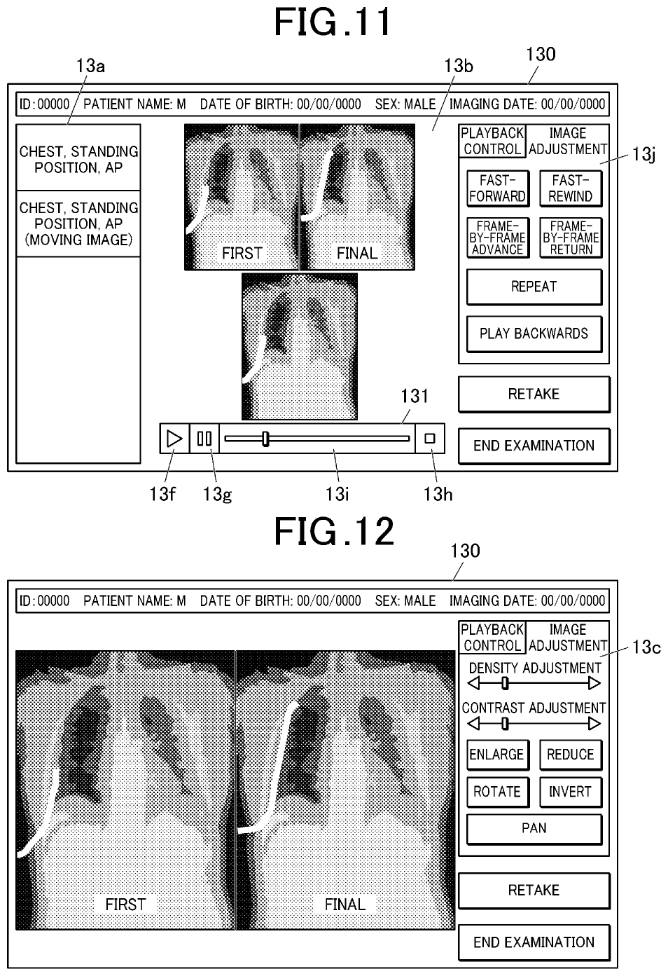

As shown in FIG. 11, when displaying the moving image in step S27 of the above-described embodiment, the controller 101 may display the first frame and the final frame side by side along with the moving image. In this way, the user can easily check the first frame and the final frame even after the moving image is ready to be checked. A key frame may also be displayed.

### Modification Example 3

The controller 101 may switch whether to perform the display of step S25 in FIG. 6, on the basis of at least one of the frame rate of the moving image, the image size, or the communication speed of the communication part 105. In other words, the controller 101 may switch whether to display the first frame and the final frame by the display part 103 based on at least one of the frame rate of the moving image, the image size, or the communication speed of the communication part 105.

For example, the actual transfer rate from the FPD 3 varies with parameters such as the frame rate during a moving image capture, the image size, and the wireless transfer speed (bps) used. Therefore, the controller 101 determines whether to perform the display of step S25 based on these parameters. For example, the controller 101 estimates the actual transfer rate on the basis of the above-described parameters. If the estimated transfer rate is lower than a predetermined threshold value, the controller 101 turns on the display of step S25. In other words, if the estimated transfer rate is lower than a predetermined threshold value, the controller 101 determines that there is a transfer delay and turns on the display of step S25. If the estimated transfer rate is higher than or equal to the predetermined threshold value, the controller 101 determines that there is no transfer delay and turns off the display of step S25. The threshold value can be set by the user through operation of the operation part 102.

### Modification Example 4

The controller 101 may notify the user of whether any of frames comprising a moving image have not yet been transferred from the FPD 3 or whether transfer of all frames has been completed. For example, the controller 101 displays the progress status such as “transfer in progress” and “transfer completed” in a predetermined area of the imaging screen 130. Alternatively, the controller 101 may display the number of received frames relative to the number of to-be-received frames in a predetermined area of the imaging screen 130. Alternatively, the controller 101 may disable pressing the moving image playback button 13f when there are untransferred frames, and enable pressing the moving image playback button 13f when all the frames have been transferred. In this way, the user can easily understand whether the transfer is currently in progress or has been completed.

### Modification Example 5

The controller 101 may change the display on the display part 103 when all frames have been transferred, depending on whether there is an outstanding imaging order for the same examination. For example, if there is no outstanding imaging order, the controller 101 shifts to moving image display upon completion of transfer of all frames. If there is an outstanding imaging order, the controller 101 highlights the outstanding imaging order in the imaging order list 13a, and displays a message such as “IMAGE CAPTURE READY” in the image display field 13b to transition to imaging of the outstanding imaging order. The user may be able to set how the screen transits at the time of completion of transfer of all frames by operating the operation part 102 before image capture.

### Modification Example 6

The next image capture will not be performed until all frames have been transferred. Therefore, as shown in FIG. 12, in step S25, the controller 101 may hide the imaging order list 13a, and use the area including the area for the imaging order list 13a as the image display field 13b to display the first frame and the final frame in larger size. This allows the first frame and the final frame to be displayed in an easily viewable manner. When the transfer of all the frames is completed, the controller 101 displays the imaging order list 13a.

### Modification Example 7

In the above-described embodiment, when the moving image capture is completed, the first (beginning) frame and the final frame are displayed side by side or in a switching manner in the image display field 13b of the imaging screen 130 in step S25 of FIG. 6. In other words, in step S25, the first frame from the beginning, which is a specific frame in the former portion of the series of frames constituting the moving image, and the first frame from the end, which is a specific frame in the latter portion, are displayed in the image display field 13b of the imaging screen 130. However, even if the specific frame in the former portion is the second frame or the third frame from the beginning and the specific frame in the latter portion is the second frame or the third frame from the last, the user can still check how the subject H has changed during the moving image capture. Therefore, the specific frame in the former portion displayed in step S25 may be the m-th frame from the beginning (m is a natural number), and the specific frame in the latter portion may be the n-th frame from the end (n is a natural number). As used herein the terms “the former portion” and “the latter portion” are not limited to the first half and the second half when all frames are divided in half. They mean the first portion and the remaining portion when all frames are divided into two at an arbitrary frame. The arbitrary frame indicates, for example, a frame between the frame displayed at the timing of the irradiation stop and the final frame.

As described above, the controller 101 of the mobile radiographic imaging apparatus 10 displays by the display part 103, as still images, the specific frame in the former portion and the specific frame in the latter portion of the series of frames constituting the moving image acquired from the FPD 3.

Therefore, the user can check how the subject H has changed during the moving image capture at an early stage after the completion of the image capture.

For example, the controller 101 displays the first frame from the beginning and the first frame from the end by the display part 103, so that the user can check how the subject H changed at the beginning and the end of the moving image capture at an early stage after the completion of the image capture.

In addition, for example, since the controller 101 displays the specific frame in the former portion and the specific frame in the latter portion on the same screen by the display part 103, the user can easily check a change between the former and latter portion frames.

Alternatively, since the controller 101 displays the former portion specific frame and the latter portion specific frame in a switching manner by the display part 103, the user can easily check a change between the former and latter portion frames.

The controller 101 further displays a key image which is an image of a frame satisfying a predetermined condition among a series of a plurality of frames of the moving image by the display part 103. Therefore, in addition to the change between the former and latter portion frames, the user can easily check the key image.

Furthermore, after the moving image capture is completed, the controller 101 displays the former portion specific frame and the latter portion specific frame by the display part 103. For example, during the moving image capture, the controller 101 displays the frames in the capturing order, starting with the first frame. When the image capture is complete, the controller 101 displays the former portion specific frame and the latter portion specific frame. Therefore, the user can check the frames obtained by image capture while the image capture is in progress, and can check the former portion specific frame and the latter portion specific frame at an early stage after the completion of the image capture.

The FPD 3 transfers the images of frames in the capturing order during the moving image capture. In response to the completion of the moving image capture, the FPD 3 stops transferring images in the capturing order and transfers the image of the specific frame in the latter portion. Therefore, even if the transfer takes time, the specific frame in the latter portion can be displayed at an early stage after the completion of the image capture.

The controller 101 notifies the user of whether there is an untransferred frame among the frames constituting the moving image or whether the transfer of all the frames has been completed. Therefore, the user can easily grasp whether the transfer of all the frames of the moving image has been completed.

The controller 101 determines whether to display the former portion specific frame and the latter portion specific frame by the display part 103, based on at least one of the frame rate of the moving image, the image size, or the wireless communication speed used. Therefore, if there is no delay in the transfer from FPD 3, the moving image can be displayed without displaying the still images of the former portion specific frame and the latter portion specific frame.

When displaying the former portion specific frame and the latter portion specific frame by the display part 103, the controller 101 performs, on the former portion specific frame and the latter portion specific frame, image processing that is different from the image processing performed on the series of a plurality of frames when displaying the moving image by the display part 103. Therefore, for example, by emphasizing the areas that the user should pay attention to, the user can easily recognize a change in the area of interest during the moving image capture.

Although embodiments of the present invention have been described and illustrated in detail, the disclosed embodiments are made for purposes of illustration and example only and not limitation. The scope of the present invention should be interpreted by terms of the appended claims.

Further, in the above description, an example in which a hard disk, a semiconductor nonvolatile memory, or the like is used as a computer-readable medium of the program according to the present invention has been disclosed, but the present invention is not limited to this example. Other applicable computer-readable media include portable recording media such as CD-ROM. In addition, a carrier wave is also applied as a medium for providing data of the program according to the present invention via a communication line.

The detailed configuration and detailed operation of the mobile radiographic imaging apparatus can also be appropriately changed without departing from the spirit and scope of the present invention.

The entire disclosure of Japanese Patent Application No. 2023-088486 filed on May 30, 2023, is incorporated herein by reference in its entirety.

What is claimed is:

1. A mobile radiographic imaging apparatus, comprising:
a hardware processor that acquires a moving image composed of a series of frames from an imaging part that captures the moving image by irradiating a subject with radiation; and
a display part that displays a display image based on the moving image,
wherein
the display part displays a first specific frame in a former portion of the series of frames and a second specific frame in a latter portion of the series of frames as still images.

2. The mobile radiographic imaging apparatus according to claim 1, wherein the first specific frame in the former portion is a m-th frame from a beginning, where m is a natural number, and the second specific frame in the latter portion is a n-th frame from an end, where n is a natural number.

3. The mobile radiographic imaging apparatus according to claim 2, wherein m=1 and n=1.

4. The mobile radiographic imaging apparatus according to claim 1, wherein the display part displays the first specific frame in the former portion and the second specific frame in the latter portion on a same screen.

5. The mobile radiographic imaging apparatus according to claim 1, wherein the display part displays the first specific frame in the former portion and the second specific frame in the latter portion one at a time on a screen by switching between the first specific frame in the former portion and the second specific frame in the latter portion.

6. The mobile radiographic imaging apparatus according to claim 1, wherein the display part further displays a key image that is a frame satisfying a predetermined condition among the series of frames.

7. The mobile radiographic imaging apparatus according to claim 1, wherein the display part displays the first specific frame in the former portion and the second specific frame in the latter portion after completion of the capturing of the moving image.

8. The mobile radiographic imaging apparatus according to claim 7, wherein the display part displays frames in a capturing order from a first frame during the capturing of the moving image, and displays the first specific frame in the former portion and the second specific frame in the latter portion when the capturing of the moving image is completed.

9. The mobile radiographic imaging apparatus according to claim 1, wherein the imaging part transfers images of the series of frame in capturing order during the capturing of the moving image, and stops transferring the images in the capturing order and transfers an image of the second specific frame in the latter portion based on completion of the capturing of the moving image.

10. The mobile radiographic imaging apparatus according to claim 9, wherein the imaging part resumes transferring the images in the capturing order after the transfer of the image of the second specific frame in the latter portion is completed.

11. The mobile radiographic imaging apparatus according to claim 1, wherein the hardware processor notifies a user of whether there is a frame among the series of frames constituting the moving image that has not been transferred from the imaging part or transfer of all the series of frames has been completed.

12. The mobile radiographic imaging apparatus according to claim 1, wherein the hardware processor determines whether to display the first specific frame in the former portion and the second specific frame in the latter portion on the display part based on at least one of a frame rate of the moving image, an image size of the moving image, or a speed of wireless communication used.

13. The mobile radiographic imaging apparatus according to claim 1, comprising an image processor that, when the first specific frame in the former portion and the second specific frame in the latter portion are displayed by the display part, performs image processing on the first specific frame in the former portion and the second specific frame in the latter portion, the image processing being different from image processing performed on the series of frames when the moving image is displayed by the display part.

14. A display method performed by a mobile radiographic imaging apparatus, comprising;
acquiring a moving image composed of a series of frames from an imaging part that captures the moving image by irradiating a subject with radiation; and
displaying a display image based on the moving image by a display part,
wherein,
in the displaying, a first specific frame in a former portion of the series of frames and a second specific frame in a latter portion of the series of frames are displayed as still images by the display part.

15. A non-transitory computer-readable recording medium storing a program that causes a computer of a mobile radiographic imaging apparatus to function as:
an image acquirer that acquires a moving image composed of a series of frames from an imaging part that captures the moving image by irradiating a subject with radiation; and
a display part that displays a display image based on the moving image,
wherein
the display part displays a first specific frame in a former portion of the series of frames and a second specific frame in a latter portion of the series of frames as still images.
